(19) United States
(12) Patent Application Publication
(10) Pub. No.: US 2010/0233669 A1
(43) Pub. Date: Sep. 16, 2010

(54) BIOBURDEN-REDUCING ANTIBIOTIC COMPOSITION AND METHOD OF USE

(75) Inventors: Steven Walsh, Marietta, GA (US); Joseph Hamby, Woodstock, GA (US); Chad Chinich, Atlanta, GA (US)

Correspondence Address: SUTHERLAND ASBILL & BRENNAN LLP, 999 PEACHTREE STREET, N.E., ATLANTA, GA, 30309, US

(73) Assignee: CRYOLIFE, INC., Kennesaw, GA (US)

(21) Appl. No.: 12/721,796

(22) Filed: Mar. 11, 2010

Related U.S. Application Data
(60) Provisional application No. 61/159,321, filed on Mar. 11, 2009.

(51) Int. Cl. A01N 1/02; C12N 5/00

(52) U.S. Cl. 435/1.1; 435/325

(57) ABSTRACT

An antibiotic composition is provided for use in decontaminating biological tissue, for example, graft tissues for transplantation or preparation for terminal sterilization. The antibiotic composition is a physiological solution includes a lantibiotic, such as nisin. The antibiotic solution is effective to substantially inhibit bacterial growth of at least one type of gram-positive bacteria while substantially maintaining the physiological characteristics of the tissue. The method may include contacting the tissue with the antimicrobial composition at a temperature and for a period of time effective to substantially inhibit bacterial growth of the at least one type of gram-positive bacteria while substantially maintaining the physiological characteristics of the tissue.

### CROSS-REFERENCE TO RELATED APPLICATIONS

This application claims priority under 35 U.S.C. §119(e) to U.S. Provisional Application No. 61/159,321, filed Mar. 11, 2009, the disclosure of which is incorporated herein by reference.

### FIELD OF THE INVENTION

This invention is generally in field of compositions and methods for decontaminating biological tissues. More specifically, it relates to bioburden-reducing, antimicrobial compositions and methods for decontaminating allograft tissues for transplantation or preparation for terminal sterilization.

### BACKGROUND OF THE INVENTION

Human allograft and other animal-derived, tissue-based implantable materials undergo a processing procedure, which may include procurement, transportation, decontamination, freezing, storage, thawing, terminal sterilization, and transplantation steps.

With respect to the decontamination step, antibiotic compositions for microbial decontamination of tissue are known in the art. In particular, several antibiotic compositions are known which contain a plurality of antibacterial agents and a single antifungal agent (amphotericin B or, occasionally, nystatin). See, e.g., Watts et al., Ann. Thorac. Surg., 21:230-36 (1976); Strickett et al., Pathology, 15:457-62 (1983); Armiger et al., Pathology, 15:67-73 (1983); Kirklin & Barratt-Boyes, Cardiac Surgery, 421-22 (1986); Heacox et al., in Cardiac Valve Allografts 1962-1987, 37-42 (Yankah et al. eds. 1988); Angell et al., J. Thorac. Cardiovasc. Surg., 98:48-56 (1989); Lange and Hopkins, in Cardiac Reconstruction With Allograft Heart Valves, 37-63 (Hopkins ed. 1989); U.S. Pat. No. 4,890,457; U.S. Pat. No. 4,695,536; and PCT Application WO 92/12632.

U.S. Pat. No. 5,741,782 describes an antibiotic composition which is effective for decontaminating and inhibiting the growth of various bacteria and fungi on cryopreserved transplant tissue. Despite its effectiveness, allograft tissues may still be occasionally rejected for bacterial contamination. The most common gram-positive bacteria present in allograft tissue rejects include S. aureus, S. epidermidis, E. faecalis, P. acnes, and S. anginosus. As such, it would be desirable to provide new antibiotic compositions and treatment methods which would reduce the frequency of allograft rejections. Additionally, new antimicrobial compositions with functionality not requiring active metabolism of the target bacteria (such as C. sporogenes) would be advantageous.

### SUMMARY OF THE INVENTION

In one aspect, an antibiotic composition for decontaminating a biological tissue is provided. The composition may comprise a solution comprising a lantibiotic in an amount effective to substantially inhibit bacterial growth of at least one type of gram-positive bacteria. The solution may be compatible with the biological tissue, such that when the solution is in contact with the biological tissue, the physiological characteristics of the biological tissue are substantially maintained.

In another aspect, a method is provided for decontaminating a biological tissue or cells. The method may comprise contacting the tissue or cells with an antibiotic composition comprising a lantibiotic for a period effective to substantially inhibit bacterial growth of at least one type of gram-positive bacteria.

In another aspect, a method is provided for preparing an allograft tissue for transplantation. The method may comprise contacting the allograft tissue with an antibiotic composition comprising a lantibiotic for a period effective to substantially inhibit bacterial growth of at least one type of gram-positive bacteria.

In another aspect, an aqueous solution is provided for treating an allograft tissue. The solution may comprise nisin in a concentration of about 1 mg per ml of solution or about 1070 IU per ml of solution; vancomycin in a concentration of about 48 μg per ml of solution; imipenem in a concentration of about 93 μg per ml of solution; amikacin in a concentration of about 36 μg per ml of solution; and amphotericin B in a concentration of about 4 μg per ml of solution.

In another aspect, a method is provided for preparing an allograft tissue for transplantation. The method may comprise contacting the allograft tissue with a tissue compatible antibiotic composition for a period effective to substantially inhibit bacterial growth of at least one type of gram-positive bacteria by killing the at least one type of gram-positive bacteria under conditions wherein the at least one type of gram-positive bacteria is substantially metabolically inactive.

In another aspect, a method is provided for reducing bioburden on an allograft or xenograft tissue in coordination with a terminal sterilization procedure. The method may comprise contacting the allograft or xenograft tissue with an antibiotic composition comprising a lantibiotic, such as nisin, for a period effective to substantially reduce the sterilization dose required in the terminal sterilization procedure to render the allograft or xenograft tissue sterile; and sterilizing the allograft or xenograft tissue in the terminal sterilization procedure to render the allograft or xenograft tissue sterile.

### DETAILED DESCRIPTION OF THE INVENTION

It has been discovered that nisin is an effective and tissue compatible antibacterial agent for use in treating tissues used for transplantation into mammalian patients, particularly humans, in need of allografts and the like. Nisin provides a targeted functionality specifically against gram positive bacteria by creating pores in the bacterial cell walls, leading to bacterial cell death, yet the pore-forming action of nisin does not occur with mammalian cells. The activity of nisin is biochemical in nature, and unlike other antibiotic compounds does not require cellular metabolism or growth to be effective, and thus is rapidly effective against even slow growing or static microbes. Nisin has been found to be particularly effective to reduce gram positive and more specifically anaerobic gram positive (e.g., Clostridium sp., P. acnes) bioburden on allograft tissues. Nisin has also been found to be an effective sporistatic agent for preventing the outgrowth of spores and subsequent increase in bioburden on allograft tissue. In addition, nisin has been found to be effective at reducing biofilm and biofilm producing gram positive bacteria (e.g., P. acnes and S. anginosis). Surprisingly, nisin was found to be significantly more effective in treating certain types of gram positive bacteria in allograft applications than traditional antibiotics that are more commonly used to combat gram positive bacteria in allograft and other applications involving human tissue.

Without being bound by any theory, it is believed that the enhanced effectiveness of nisin in bioburden-reducing compositions for allograft tissue may be the result of its unique killing mechanism, which may not be sensitive to environmental conditions such as temperature and oxygen conditions. Most conventional antibiotics used for decontaminating gram positive bacteria in allograft and human tissue applications intervene during cell division or when the bacteria are metabolically active. Accordingly, these conventional antibiotics may require temperature and oxygen conditions that support cell division and metabolism. These conventional antibiotics may not be effective at killing bacteria that are reproductively or metabolically dormant at the time the bacteria are exposed to the antibiotic. Moreover, some bacterial strains may have developed resistance to conventional antibiotics. Because, nisin's mechanism for killing bacteria is different than that of most conventional antibiotics, it may be even more effective against these antibiotic-resistant bacterial strains. Also, because nisin's kill mechanism is different than that of conventional antibiotics used in bioburden-reducing cocktails for allograft tissue, nisin is unlikely to exert any counter-acting effects on the other active agents used in the cocktail. Accordingly, nisin and lantibiotics having similar biochemical activity advantageously can improve the net safety profile and the processing yield of allograft tissue.

In one aspect, an antibiotic composition is provided for use in decontaminating biological tissue, for example, graft tissues for transplantation. In one embodiment, the antibiotic composition is a physiological solution comprising a lantibiotic, such as nisin. The antibiotic solution is effective to substantially inhibit bacterial growth of at least one type of gram-positive bacteria, while substantially maintaining the physiological characteristics of the tissue.

In another aspect, a method is provided for reducing bioburden on an allograft or xenograft tissue in coordination with a terminal sterilization procedure. The method may comprise contacting the allograft or xenograft tissue with an antibiotic composition comprising a lantibiotic, such as nisin, for a period effective to substantially reduce the sterilization dose required in the terminal sterilization procedure to render the allograft or xenograft tissue sterile; and sterilizing the allograft or xenograft tissue in the terminal sterilization procedure to render the allograft or xenograft tissue sterile. The terminal sterilization procedure may comprise, for example, subjecting the allograft or xenograft tissue to ionizing radiation. The allograft or xenograft tissue may be contacted with the lantibiotic for a period effective to substantially inhibit bacterial growth of at least one type of gram-positive bacteria. In such a method, the selection of terminal sterilization dose parameters may be directly correlated to the residual bioburden on the allograft or xenograft tissue. For example, when using ionizing radiation (e.g., Ebeam or gamma irradiation) a bioburden of 1000 cfu/device may require a minimum dose of 25 KGy (VDmax25) to provide assurance of sterility. If the step of contacting the allograft or xenograft tissue with the antibiotic composition yielded a residual bioburden of 1.5 cfu/device, the same sterility assurance level can be achieved with 15 KGy (VDmax15) dose. This could extend the application of terminal sterilization technologies to allograft and xenograft tissues that might otherwise be considered inappropriate due to concerns over negative effects to structural integrity or other physiological characteristics.

In exemplary embodiments, the antibiotic composition further comprises at least one antifungal agent. In certain embodiments, the antifungal agent comprises a polyene, such as Amphotericin B.

In some embodiments, the antibiotic composition further comprises antibacterial agents in addition to nisin. In certain embodiments, the antibiotic composition comprises nisin with one or more other antibacterial agents selected from glycopeptides, beta lactam, aminoglycoside, or a combination thereof.

In another aspect, methods of decontaminating a tissue for transplantation are provided. The method includes contacting the tissue with the antimicrobial composition. In a preferred embodiment, the tissue is contacted with the antimicrobial composition at a temperature and for a period effective to substantially inhibit bacterial growth of at least one type of gram-positive bacteria while substantially maintaining the physiological characteristics of the tissue.

Using the antibiotic compositions, many tissues that would have to be rejected because of yeast or bacterial contamination can be used for transplantation. This is a significant achievement in view of the scarcity of tissue available for transplantation.

As used herein, the term “amounts effective,” “effective amount,” or the like as used in reference to one or more of the antimicrobial agents means that the agent(s) is/are present at a sufficient concentration such that the composition substantially inhibits yeast and/or bacterial growth but does not substantially negatively alter physiological characteristics of the tissue which would affect the tissue's suitability for use in an allograft application. Suitability may be determined by evaluating physiological characteristics of the tissue including, but not limited to, viability, biomechanics, denaturation temperature, and microscopic evaluation of the tissue. Thus, “effective amounts” can be determined by dose response testing as is known in the art using standard microbiological tests and viability tests such as those known in the art or described below. Preferably the agents are present in the composition in amounts which are cidal for yeasts and/or bacteria frequently isolated from tissue.

As used herein, the term “substantially inhibits” means that the composition completely inhibits yeast and/or bacterial growth in at least 90%, preferably at least 99%, most preferably at least 99.9%, of the tissues treated with the composition. The term “substantially inhibits” further encompasses various mechanisms for inhibiting bacterial growth including, but not limited to, interrupting the metabolic activity of the bacteria or killing the bacteria. “Completely inhibits yeast and bacterial growth” means that yeast and bacterial growth are not detectable by standard microbiological assays after the tissue has been treated with the composition.

As used herein the term “substantially maintaining the physiological characteristics of the tissue” means that the composition does not aversely affect the physiological characteristics of the tissue that render the tissue suitable for use in reconstruction, repair or replacement. As such, the physiological characteristics that are maintained depend on the physiological characteristics of the tissue subject to the decontamination treatment with the composition. For example, for allograft tissues having viable cells that are to remain viable during transplantation, the term “substantially maintaining the physiological characteristics of the tissue” further encompasses substantially maintaining the viability of the cells of the tissue. For allograft tissues that do not have viable cells, such as decellularized tissue grafts, the term “substantially maintaining the physiological characteristics of the tissue” primarily encompasses maintaining the biomechanical properties of the tissue without denaturing collagen, elastin, and other protein components of the tissue structure.

In some embodiments, as explained above, the composition is effective at substantially inhibiting the growth of at least one strain of gram positive bacteria while substantially maintaining the viability of the tissue. Viability can be measured in a number of ways. In one embodiment, the tissue is incubated with a radioactively-labeled amino acid, and the incorporation of the amino acid into proteins is monitored by counting disintegrations per minute (DPM) per unit of tissue. Accordingly, as used herein, the term “substantially maintaining the viability” means that tissue that has been treated with the composition incorporates at least about 85% of the DPM per unit tissue, as compared to tissue that is not treated with the composition.

In one aspect, an antibiotic composition is provided for decontaminating biological tissue. The tissue may be an allograft or other tissue suitable for transplantation. The antibiotic composition may be an antibiotic solution comprising an antimicrobial polypeptide. More preferably, the antibiotic solution comprises a bacteriocin, and more preferably a lantibiotic, such as nisin in an appropriate solvent. Representative examples of suitable solvents include, but are not limited to, physiological saline, and phosphate buffered saline. Other solutions/media known in the art for storing or treating cellular or tissue based materials may be used. The solvent preferably has a pH between 6 and 8. The nisin is provided in sufficient concentration and in an effective amount such that the antibiotic solution is effective at substantially inhibiting bacterial growth of at least one type of gram-positive bacteria while substantially maintaining the physiological characteristics of the tissue.

Nisin is a polycyclic peptide, and it is active against various gram-positive bacteria. It has been discovered that Nisin is particularly effective against S. aureus, S. epidermidis, E. faecalis, P. acnes, C. sporogenes and S. anginosus. Further, nisin has been found to be generally effective for substantially inhibiting bacterial growth of various gram-positive bacteria, including S. aureus, S. epidermidis, E. faecalis, P. acnes, C. sporogenes and S. anginosus on a transplant tissue while substantially maintaining the viability of the tissue when used in a concentration range of 250 to 10,000 IU/mL, more preferably in a range of 500 to 1200 IU/mL, and most preferably in a concentration of about 1000 IU/mL.

In certain embodiments, the antibiotic composition comprising nisin further includes one or more antifungal agents. In a preferred embodiment, the antifungal agent is a polyene, such as amphotericin B. Other antifungal agents known in the art may be used with or in place of amphotericin B. The use of amphotericin B is particularly effective for substantially inhibiting yeast growth.

Suitable concentrations of amphotericin B can be determined by dose response testing as is known in the art using standard microbiological tests and viability tests. Amphotericin B alone at concentrations of ≧1 ppm is capable of high effectiveness against yeasts and does not negatively effective the tissue viability (even though these concentrations would be cytotoxic to kidney nephrons, not fibroblasts).

A concentration of from about 1.0 μg/ml to about 4.0 μg/ml of amphotericin B is preferred for use in one embodiment of an antibiotic composition for reducing yeast contamination on cardiovascular tissues. Other concentrations may also be suitably effective for use with other tissues or in other tissue decontamination processes.

In certain embodiments, the antibiotic solution further includes antibacterial agents effective against a wide range of bacteria, including gram-negative, gram-positives aerobic and anaerobic bacteria. In addition, the antibacterial agents preferably are chosen so that the combination of agents is effective against bacteria commonly found to contaminate the tissue being treated. Many such bacteria are known (e.g., staphylococci, streptococci and propionibacteria) and others can be identified by standard microbiological tests. Thus, broad spectrum antibacterial agents from two or more families are preferred. For preferred tissue applications and transplantations, the selected combination of antibacterial agents should not substantially effect the physiological characteristics of the tissue being treated.

In a preferred embodiment, antibacterial agents are chosen from the following families: cephalosporins, glycopeptides, aminoglycosides, lincosamides, quinalones, beta-lactams, and rifamycins. More preferably, the combination of antibacterial agents comprises Nisin, vancomycin and imipenem, and most preferably nisin, vancomycin, imipenem and amikacin. For the decontamination of cardiovascular tissues, a combination of about 1000 IU/ml Nisin, about 44 μg/ml vancomycin, about 83 μg/ml imipenem and about 33 μg/ml amikacin is preferred.

Imipenem is a beta-lactam antibiotic. It is active against most aerobic gram-positive and gram-negative bacteria and most anaerobic gram-positive and gram-negative bacteria.

Amikacin (amikacin sulfate) is another broad-spectrum antibiotic that typically provides effectiveness against both gram-positive and gram-negative bacteria in concentrations 2 to 3, and more preferably 4 to 8, times the minimum inhibitory concentration for gram-positive bacteria.

Vancomycin is a tricyclic glycopeptide. It is active against many gram-positive organisms, including staphylococci, streptococci, enterococci, Clostridium and Corynebacterium. It is inactive against gram-negative bacteria.

Although imipenem, amikacin, and vancomycin show good broad spectrum activity, these antibiotics are only effective under certain environmental conditions, particularly environmental conditions which allow microbial growth. For example, vancomycin, although clinically effective against Clostridium sp. is totally ineffective against the anaerobe under aerobic conditions. This limitation is based on the antibiotics' mechanism of interrupting the metabolic activity of the bacteria. Nisin, because of its unique biochemical activity, is effective under a broad range of environmental conditions, without regard to the metabolic activity of the bacteria.

In a preferred embodiment, the concentrations of the antibacterial agents are chosen to be at least 2 to 3 times, and more preferably 4 to 8 times, the minimum inhibitory concentrations for the targeted bacteria as determined by standard microbiological sensitivity assays. Within these parameters, the concentrations of antibacterial agents can be adjusted as a result of dose response testing on tissue using standard microbiological tests and viability tests.

From the foregoing, it can be seen that a preferred embodiment of the present antibiotic compositions contains nisin, amphotericin B, vancomycin, imipenem, and amikacin. For decontamination of cardiovascular tissues, antibiotic compositions containing about 1-5 μg/ml amphotericin B, about 40-60 μg/ml vancomycin, about 70-120 μg/ml imipenem, about 30-50 μg/ml amikacin, and about 850-1150 IU/ml nisin are preferred.

In a particularly preferred embodiment, the composition comprises: 4 μg/ml amphotericin B, 48 μg/ml vancomycin, 92 μg/ml imipenem, 36 μg/ml amikacin, and 1070 IU/ml nisin.

In another aspect, a method of decontamination a biological tissue or inhibiting the growth of bacteria in a transplant tissue is provided. In one embodiment, the method includes the step of contacting the tissue with an antibiotic composition as described above. A variety of tissues may be decontaminated in this manner. Representative examples of tissues suitable with the present antimicrobial compositions and methods include heart valves, pericardium, vessels, and musculoskeletal connective tissue. As used herein, the term “musculoskeletal connective tissue” includes tissue such as tendons, ligaments and menisci.

In a preferred embodiment of the method, the tissue is contacted with the antibiotic composition at a temperature and for a period of time effective to substantially inhibit yeast and bacterial organisms while substantially maintaining the physiological characteristics of the tissue. Such times and temperatures can be determined empirically as is known in the art. It has been found that heart valves can be effectively decontaminated by incubating them in a Nisin-containing antibiotic composition for 10-48 hours at a temperature of 2°-37° C. Other allograft tissues may be effectively decontaminated by contacting the allograft tissue in a Nisin-containing antibiotic composition for the same period of time and temperature. As used herein, the term “contact” is broadly used to describe any method of applying the composition to a tissue including, but not limited to, spraying the composition onto the tissue and submerging the tissue into a solution comprising the composition.

In yet another aspect, a method is provided for preparing and delivering a tissue for transplantation. First, the tissue is procured from a donor. Next, in a typical embodiment, the tissue is dissected to separate the tissue component to be used in the transplantation from tissue material that will not be part of the transplantation or that must otherwise be separated from the tissue component prior to transplantation. For example, if the tissue component comprises a heart valve, the heart valve may be dissected as described in U.S. Pat. No. 4,890,457.

Then, in one embodiment, the tissue component is subjected to the aforementioned decontamination treatment with a bioburden-reducing antibiotic composition, which comprises an effective amount of Nisin. For example, the tissue component may be submerged in a solution comprising: 4 μg/ml amphotericin B, 48 μg/ml vancomycin, 92 μg/ml imipenem, 36 μg/ml amikacin, and 1070 IU/ml nisin in an appropriate physiologic media.

In one embodiment, the tissue component is then packaged, cryopreserved and stored as described in U.S. Pat. No. 4,890,457. The tissue component may be frozen gradually to −80° C. The tissue component may then be shipped in its frozen state.

In a typical embodiment, the tissue component is thawed and rinsed before transplantation. In one embodiment, the tissue component is immersed in a hypertonic solution comprising electrolytes and dextrose, such as Lactated Ringer's to compensate for the loss of extracellular fluids during cryopreservation.

Finally, the tissue component is transplanted into a patient using suitable grafting and surgical techniques known in the art.

The present invention is further illustrating by the following non-limiting examples.

### Example 1

This example describes the preparation of one embodiment of an antibiotic composition for decontaminating tissue for transplantation.

A nisin stock solution was prepared by dissolving nisin (Sigma-Aldrich) in WFI (Water-For-Injection) to a concentration of 35 mg of nisin per ml of solution (35,000 IU/mL). The stock solution was filter sterilized and stored at 2-8° C.

A stock antimicrobial solution was produced by adding 380 ml of the nisin stock solution to 420 ml of a second antibiotic solution. The second antibiotic solution comprised vancomycin in a concentration of 650 μg per ml of solution, imipenem in a concentration of 1250 μg per ml of solution, amikacin in a concentration of 489 μg per ml of solution, and amphotericin B in a concentration of 54 μg per ml of solution dissolved in Dulbecco's Modified Eagle Media (DMEM). The stock antimicrobial solution was frozen in functional volumes and stored at a temperature less than −70° C.

A final antibiotic composition solution was then prepared by diluting 1 ml of the stock antimicrobial solution to 13.5 ml with phosphate buffered saline. The final solution comprised nisin in a concentration of about 1 mg per ml (1070 IU/mL) of solution, vancomycin in a concentration of about 48 μg per ml of solution, imipenem in a concentration of about 93 μg per ml of solution, amikacin in a concentration of about 36 μg per ml of solution, and amphotericin B in a concentration of about 4 μg per ml of solution.

### Example 2

This examples demonstrates the kill effectiveness of a nisin solution tested against a panel of gram positive microbes identified most frequently in allograft decontamination rejects (S. aureus, S. epidermidis, E. faecalis, P. acnes, and S. anginosus).

The nisin solution was prepared by adding nisin (Sigma-Aldrich) to DMEM to produce a solution comprising Nisin in a concentration of about 191 ppm.

A 10⁸ cfu/ml S. aureus inoculum was used to prepare the S. aureus test samples. Count plates were prepared by plating 100 μl of the 10⁻⁵ and 10⁻⁶ dilutions on duplicate Triptic Soy Agar (“TSA”) plates. Four conical tubes were prepared and labeled in duplicate for the control and the test. A 19 ml volume of nisin solution was transferred to each test tube, and 19 ml of Solution B was transferred to each control tube. The four tubes were then inoculated with 1 ml of 10⁸ inoculum (5×10⁶ cfu/ml final concentration) and inverted 2-3 times to mix the solution. Following inoculation, a 1 ml sample was removed from each treatment tube and serially diluted to 10⁻⁶. The 10⁻⁴ to 10⁻⁶ dilutions from each tube were filtered and plated on TSA plates and placed in the incubator at 35-39° C. The test and control tubes were placed back into the incubator at 35-39° C. for 24 hours, and at the 7-hour and 24-hour treatment intervals, a 1 ml sample from each tube was filtered and plated to provide a 10° dilution. A second 1 ml sample was diluted to 10⁻⁶, and dilutions of 10⁻¹ to 10⁻⁶ from each tube were filtered and plated. All plates were placed in the incubator for 3-5 days and colony counts were reported for each dilution.

The procedure was repeated with four other organisms (S. epidermidis, E. faecalis, P. acnes, and S. anginosus) using the appropriate culture plate for each organism.

The results from each dilution were calculated by averaging the colony counts and multiplying the average by the dilution factor. The result from the lowest dilution with the highest count between 25-250 cfu was reported for each solution type and time point. The results are provided in TABLES 1-5.

TABLE 1

| | Dilution | 10⁰ ml | 10⁻¹ ml | 10⁻² ml | 10⁻³ ml | 10⁻⁴ ml | 10⁻⁵ ml | 10⁻⁶ ml |
|---|---|---|---|---|---|---|---|---|
| | Multiplier | 2E+01 | 2E+02 | 2E+03 | 2E+04 | 2E+05 | 2E+06 | 2E+07 |
| S. aureus | T-0 Control 1 | | | | | TNTC | 42 | 4 |
| | T-0 Control 2 | | | | | TNTC | 33 | 1 |
| | AVG Count | | | | | | 37.5 | 2.5 |
| | Results | 0.00E+00 | 0.00E+00 | 0.00E+00 | 0.00E+00 | 0.00E+00 | 7.50E+07 | 5.00E+07 |
| | T-0 Test 1 | | | | | 48 | 3 | 0 |
| | T-0 Test 2 | | | | | 49 | 3 | 1 |
| | AVG Count | | | | | 48.5 | 3 | 0.5 |
| | Results | 0.00E+00 | 0.00E+00 | 0.00E+00 | 0.00E+00 | 9.70E+06 | 6.00E+06 | 1.00E+07 |
| | T-7 Control 1 | TNTC | TNTC | TNTC | TNTC | 117 | 28 | 1 |
| | T-7 Control 2 | TNTC | TNTC | TNTC | TNTC | 60 | 0 | 0 |
| | AVG Count | | | | | 88.5 | 14 | 0.5 |
| | Results | 0.00E+00 | 0.00E+00 | 0.00E+00 | 0.00E+00 | 1.77E+07 | 2.80E+07 | 1.00E+07 |
| | T-7 Test 1 | TNTC | 61 | 8 | 6 | 0 | 0 | 0 |
| | T-7 Test 2 | TNTC | 72 | 14 | 1 | 0 | 0 | 0 |
| | AVG Count | | 66.5 | 11 | 3.5 | 0 | 0 | 0 |
| | Results | 0.00E+00 | 1.33E+04 | 2.20E+04 | 7.00E+04 | 0.00E+00 | 0.00E+00 | 0.00E+00 |
| | T-24 Control 1 | TNTC | TNTC | TNTC | TNTC | TNTC | 136 | 7 |
| | T-24 Control 2 | TNTC | TNTC | TNTC | TNTC | TNTC | 101 | 6 |
| | AVG Count | | | | | | 118.5 | 6.5 |
| | Results | 0.00E+00 | 0.00E+00 | 0.00E+00 | 0.00E+00 | 0.00E+00 | 2.37E+08 | 1.30E+08 |
| | T-24 Test 1 | TNTC | 180 | 34 | 2 | 0 | 0 | 0 |
| | T-24 Test 2 | TNTC | 243 | 48 | 3 | 0 | 0 | 0 |
| | AVG Count | | 211.5 | 41 | 2.5 | 0 | 0 | 0 |
| | Results | 0.00E+00 | 4.23E+04 | 8.20E+04 | 5.00E+04 | 0.00E+00 | 0.00E+00 | 0.00E+00 |

* TNTC—too numerous to count

TABLE 1 shows the colony counts for each S. aureus test and control sample. The count plates for S. aureus showed an average of 97 colonies at the 10⁻⁵ dilution, giving an initial inoculum concentration of 1.9×10⁸ cfu/ml. The control solution showed an average of 7.5×10⁷ cfu for the 0 hour (T-0) treatment interval. The average for the 7-hour (T-7) and 24-hour (T-24) intervals for the control solution were 1.8×10⁷ cfu and 2.4×10⁸ cfu, respectively. The total remaining viable S. aureus colonies after treatment with the 191 ppm nisin solution were 9.7×10⁶ cfu for the T-0 treatment interval, 1.3×10⁴ cfu for the T-7 treatment interval, and 4.2×10⁴ cfu at the T-24 treatment interval.

The S. aureus control showed a 1 log decrease between the T-0 and T-7 timepoints, while there was a 1 log increase from the T-0 to the T-24 timepoints. The nisin-treated S. aureus showed a 3 log decrease at the T-7 timepoint, and after a slight rebound in growth, showed a 2 log kill between the T-0 and T-24 timepoints.

TABLE 2

| | Dilution | 10⁰ ml | 10⁻¹ ml | 10⁻² ml | 10⁻³ ml | 10⁻⁴ ml | 10⁻⁵ ml | 10⁻⁶ ml |
|---|---|---|---|---|---|---|---|---|
| | Multiplier | 2E+01 | 2E+02 | 2E+03 | 2E+04 | 2E+05 | 2E+06 | 2E+07 |
| S. epidermidis | T-0 Control 1 | | | | | 141 | 19 | 3 |
| | T-0 Control 2 | | | | | 149 | 25 | 3 |
| | AVG Count | | | | | 145 | 22 | 3 |
| | Results | 0.00E+00 | 0.00E+00 | 0.00E+00 | 0.00E+00 | 2.90E+07 | 4.40E+07 | 6.00E+07 |
| | T-0 Test 1 | | | | | 55 | 2 | 0 |
| | T-0 Test 2 | | | | | 32 | 4 | 0 |
| | AVG Count | | | | | 43.5 | 3 | 0 |
| | Results | 0.00E+00 | 0.00E+00 | 0.00E+00 | 0.00E+00 | 8.70E+06 | 6.00E+06 | 0.00E+00 |
| | T-7 Control 1 | TNTC | TNTC | TNTC | TNTC | TNTC | 42 | 11 |
| | T-7 Control 2 | TNTC | TNTC | TNTC | TNTC | TNTC | 58 | 3 |
| | AVG Count | | | | | | 50 | 7 |
| | Results | 0.00E+00 | 0.00E+00 | 0.00E+00 | 0.00E+00 | 0.00E+00 | 1.00E+08 | 1.40E+08 |
| | T-7 Test 1 | 38 | 2 | 1 | 0 | 0 | 0 | 0 |
| | T-7 Test 2 | 47 | 4 | 0 | 0 | 0 | 0 | 0 |
| | AVG Count | 42.5 | 3 | 0.5 | 0 | 0 | 0 | 0 |
| | Results | 8.50E+02 | 6.00E+02 | 1.00E+03 | 0.00E+00 | 0.00E+00 | 0.00E+00 | 0.00E+00 |
| | T-24 Control 1 | TNTC | TNTC | TNTC | TNTC | TNTC | 43 | 2 |
| | T-24 Control 2 | TNTC | TNTC | TNTC | TNTC | TNTC | 58 | 0 |
| | AVG Count | | | | | | 50.5 | 1 |
| | Results | 0.00E+00 | 0.00E+00 | 0.00E+00 | 0.00E+00 | 0.00E+00 | 1.01E+08 | 2.00E+07 |
| | T-24 Test 1 | N/A | N/A | N/A | N/A | N/A | N/A | N/A |
| | T-24 Test 2 | TNTC | TNTC | 75 | 9 | 0 | 0 | 0 |
| | AVG Count | | | 75 | 9 | 0 | 0 | 0 |
| | Results | 0.00E+00 | 0.00E+00 | 1.50E+05 | 1.80E+05 | 0.00E+00 | 0.00E+00 | 0.00E+00 |

TABLE 2 shows the colony counts for each S. epidermidis test and control sample. The count plates for S. epidermidis showed an average of 26 colonies at the 10⁻⁵ dilution, giving an initial inoculum concentration of 5.2×10⁷ cfu/ml. The control solution showed an average of 2.9×10⁷ cfu for the T-0 treatment interval, 1.0×10⁸ cfu for the T-7 treatment interval, and 1.0×10⁸ cfu for the T-24 treatment interval. The total remaining viable S. epidermidis colonies after treatment with the 191 ppm nisin solution were 8.7×10⁶ cfu for the T-0 treatment interval, 8.5×10² cfu for the T-7 treatment interval, and 1.5×10⁵ cfu at the T-24 interval.

The S. epidermidis control had a 1 log increase through the T-7 timepoint, and remained at that level through the T-24 timepoint. The nisin-treated S. epidermidis showed a 4 log decrease through 7 hours, and after a rebound in growth showed an overall kill of 2 log after 24 hours.

TABLE 3

| | Dilution | 10⁰ ml | 10⁻¹ ml | 10⁻² ml | 10⁻³ ml | 10⁻⁴ ml | 10⁻⁵ ml | 10⁻⁶ ml |
|---|---|---|---|---|---|---|---|---|
| | Multiplier | 2E+01 | 2E+02 | 2E+03 | 2E+04 | 2E+05 | 2E+06 | 2E+07 |
| E. faecalis | T-0 Control 1 | | | | | 84 | 18 | 4 |
| | T-0 Control 2 | | | | | 108 | 10 | 1 |
| | AVG Count | | | | | 96 | 37.5 | 2.5 |
| | Results | 0.00E+00 | 0.00E+00 | 0.00E+00 | 0.00E+00 | 1.92E+07 | 2.80E+07 | 4.00E+07 |
| | T-0 Test 1 | | | | | 32 | 9 | 0 |
| | T-0 Test 2 | | | | | 15 | 4 | 0 |
| | AVG Count | | | | | 23.5 | 6.5 | 0 |
| | Results | 0.00E+00 | 0.00E+00 | 0.00E+00 | 0.00E+00 | 4.70E+06 | 1.30E+07 | 0.00E+00 |
| | T-7 Control 1 | TNTC | TNTC | TNTC | TNTC | 244 | 15 | 0 |
| | T-7 Control 2 | TNTC | TNTC | TNTC | TNTC | 224 | 21 | 1 |
| | AVG Count | | | | | 234 | 18 | 0.5 |
| | Results | 0.00E+00 | 0.00E+00 | 0.00E+00 | 0.00E+00 | 4.68E+07 | 3.60E+07 | 1.00E+07 |
| | T-7 Test 1 | 3 | 6 | 0 | 0 | 0 | 0 | 0 |
| | T-7 Test 2 | 6 | 5 | 0 | 0 | 0 | 0 | 0 |
| | AVG Count | 4.5 | 5.5 | 0 | 0 | 0 | 0 | 0 |
| | Results | 9.00E+01 | 1.10E+03 | 0.00E+00 | 0.00E+00 | 0.00E+00 | 0.00E+00 | 0.00E+00 |
| | T-24 Control 1 | TNTC | TNTC | TNTC | TNTC | TNTC | 44 | 5 |
| | T-24 Control 2 | TNTC | TNTC | TNTC | TNTC | TNTC | 47 | 4 |
| | AVG Count | | | | | | 45.5 | 4.5 |
| | Results | 0.00E+00 | 0.00E+00 | 0.00E+00 | 0.00E+00 | 0.00E+00 | 9.10E+07 | 9.00E+07 |
| | T-24 Test 1 | TNTC | TNTC | TNTC | TNTC | 56 | 6 | 0 |
| | T-24 Test 2 | TNTC | TNTC | TNTC | TNTC | 43 | 2 | 0 |
| | AVG Count | | | | | 49.5 | 4 | 0 |
| | Results | 0.00E+00 | 0.00E+00 | 0.00E+00 | 0.00E+00 | 9.90E+06 | 8.00E+06 | 0.00E+00 |

TABLE 3 shows the colony counts for each E. faecalis test and control sample. The count plates for E. faecalis showed an average of 41 colonies at the 10⁻⁵ dilution, giving an initial inoculum concentration of 8.2×10⁷ cfu/ml. The control solution showed an average of 1.9×10⁷ cfu for the T-0 treatment interval, 4.7×10⁷ cfu for the T-7 treatment interval, and 9.1×10⁷ cfu for the T-24 treatment interval. The total remaining viable E. faecalis colonies after the treatment with the 191 ppm nisin solution were 4.7×10⁶ cfu for the T-0 treatment interval, 9.0×10¹ cfu for the T-7 treatment interval, and 9.9×10⁶ cfu at the T-24 treatment interval.

The E. faecalis control showed less than 1 log increase at 7 hours, and the growth remained at less than 1 log through the 24 hour period. The nisin-treated E. faecalis showed a 5 log decrease in 7 hours, and after a large rebound in growth, showed a slightly higher level (less than 1 log) at 24 hours than at the original T-0 timepoint.

TABLE 4

| | Dilution | 10⁰ ml | 10⁻¹ ml | 10⁻² ml | 10⁻³ ml | 10⁻⁴ ml | 10⁻⁵ ml | 10⁻⁶ ml |
|---|---|---|---|---|---|---|---|---|
| | Multiplier | 2E+01 | 2E+02 | 2E+03 | 2E+04 | 2E+05 | 2E+06 | 2E+07 |
| P. acnes | T-0 Control 1 | | | | | 247 | 30 | 6 |
| | T-0 Control 2 | | | | | 241 | 22 | 1 |
| | AVG Count | | | | | 244 | 26 | 3.5 |
| | Results | 0.00E+00 | 0.00E+00 | 0.00E+00 | 0.00E+00 | 4.88E+07 | 5.20E+07 | 7.00E+07 |
| | T-0 Test 1 | | | | | 192 | 3 | 2 |
| | T-0 Test 2 | | | | | 203 | 9 | 3 |
| | AVG Count | | | | | 197.5 | 6 | 2.5 |
| | Results | 0.00E+00 | 0.00E+00 | 0.00E+00 | 0.00E+00 | 3.95E+07 | 1.20E+07 | 5.00E+07 |
| | T-7 Control 1 | TNTC | TNTC | TNTC | TNTC | 248 | 24 | 1 |
| | T-7 Control 2 | TNTC | TNTC | TNTC | TNTC | 228 | 21 | 4 |
| | AVG Count | | | | | 238 | 22.5 | 2.5 |
| | Results | 0.00E+00 | 0.00E+00 | 0.00E+00 | 0.00E+00 | 4.76E+07 | 4.50E+07 | 5.00E+07 |
| | T-7 Test 1 | N/A | TNTC | TNTC | 44 | 5 | 1 | 0 |
| | T-7 Test 2 | N/A | TNTC | TNTC | 38 | 4 | 0 | 0 |
| | AVG Count | | | | 41 | 4.5 | 0.5 | 0 |
| | Results | 0.00E+00 | 0.00E+00 | 0.00E+00 | 8.20E+05 | 9.00E+05 | 1.00E+06 | 0.00E+00 |
| | T-24 Control 1 | TNTC | TNTC | TNTC | TNTC | 184 | 28 | 6 |
| | T-24 Control 2 | TNTC | TNTC | TNTC | TNTC | 160 | 13 | 0 |
| | AVG Count | | | | | 172 | 20.5 | 3 |
| | Results | 0.00E+00 | 0.00E+00 | 0.00E+00 | 0.00E+00 | 3.44E+07 | 4.10E+07 | 6.00E+07 |
| | T-24 Test 1 | 3 | 11 | 1 | 2 | 1 | 0 | 0 |
| | T-24 Test 2 | 0 | 0 | 7 | 0 | 0 | 0 | 0 |
| | AVG Count | 1.5 | 5.5 | 4 | 1 | 0.5 | 0 | 0 |
| | Results | 3.00E+01 | 1.10E+03 | 8.00E+03 | 2.00E+04 | 1.00E+05 | 0.00E+00 | 0.00E+00 |

TABLE 4 shows the colony counts for each P. acnes test and control sample. The count plates for P. acnes showed an average of 112 colonies at the 10⁻⁵ dilution, giving an initial inoculum concentration of 2.2×10⁸ cfu/ml. The control solution showed an average of 4.9×10⁷ cfu for the T-0 treatment interval, 4.7×10⁷ cfu for the T-7 treatment interval, and 3.4×10⁷ cfu for the T-24 treatment interval. The total remaining viable P. acnes colonies after treatment with the 191 ppm nisin solution were 4.0×10⁷ cfu for the T-0 treatment interval, 8.2×10⁵ cfu for the T-7 treatment interval, and 3.0×10¹ cfu for the T-24 treatment interval.

The P. acnes control showed no growth between T-0 and T-7 timepoints with a less than 1 log decrease at 24 hours. The nisin-treated P. acnes showed a 2 log decrease by T-7 and a 6 log total decrease by the T-24 timepoint.

TABLE 5

| | Dilution | 10⁰ ml | 10⁻¹ ml | 10⁻² ml | 10⁻³ ml | 10⁻⁴ ml | 10⁻⁵ ml | 10⁻⁶ ml |
|---|---|---|---|---|---|---|---|---|
| | Multiplier | 2E+01 | 2E+02 | 2E+03 | 2E+04 | 2E+05 | 2E+06 | 2E+07 |
| S. anginosus | T-0 Control 1 | | | | | 39 | 2 | 0 |
| | T-0 Control 2 | | | | | 42 | 1 | 0 |
| | AVG Count | | | | | 40.5 | 1.5 | 0 |
| | Results | 0.00E+00 | 0.00E+00 | 0.00E+00 | 0.00E+00 | 8.10E+06 | 3.00E+06 | 0.00E+00 |
| | T-0 Test 1 | | | | | 0 | 0 | 0 |
| | T-0 Test 2 | | | | | 0 | 0 | 0 |
| | AVG Count | | | | | 0 | 0 | 0 |
| | Results | 0.00E+00 | 0.00E+00 | 0.00E+00 | 0.00E+00 | 0.00E+00 | 0.00E+00 | 0.00E+00 |
| | T-7 Control 1 | TNTC | TNTC | TNTC | 242 | 18 | 0 | 1 |
| | T-7 Control 2 | TNTC | TNTC | TNTC | 237 | 26 | 2 | 0 |
| | AVG Count | | | | 239.5 | 22 | 1 | 0.5 |
| | Results | 0.00E+00 | 0.00E+00 | 0.00E+00 | 4.79E+06 | 4.40E+06 | 2.00E+06 | 1.00E+07 |
| | T-7 Test 1 | 0 | 0 | 0 | 0 | 0 | 0 | 0 |
| | T-7 Test 2 | 0 | 0 | 0 | 0 | 0 | 0 | 0 |
| | AVG Count | 0 | 0 | 0 | 0 | 0 | 0 | 0 |
| | Results | 0.00E+00 | 0.00E+00 | 0.00E+00 | 0.00E+00 | 0.00E+00 | 0.00E+00 | 0.00E+00 |
| | T-24 Control 1 | TNTC | TNTC | TNTC | TNTC | 38 | 0 | 0 |
| | T-24 Control 2 | TNTC | TNTC | TNTC | TNTC | 47 | 6 | 0 |
| | AVG Count | | | | | 42.5 | 3 | 0 |
| | Results | 0.00E+00 | 0.00E+00 | 0.00E+00 | 0.00E+00 | 8.50E+06 | 6.00E+06 | 0.00E+00 |
| | T-24 Test 1 | 0 | 0 | 0 | 0 | 0 | 0 | 0 |
| | T-24 Test 2 | 0 | 0 | 0 | 0 | 0 | 0 | 0 |
| | AVG Count | 0 | 0 | 0 | 0 | 0 | 0 | 0 |
| | Results | 0.00E+00 | 0.00E+00 | 0.00E+00 | 0.00E+00 | 0.00E+00 | 0.00E+00 | 0.00E+00 |

TABLE 5 shows the colony counts for each S. anginosus test and control sample. The count plates for S. anginosus showed an average of 14 colonies at the 10⁻⁵ dilution, giving an initial inoculum concentration of 2.8×10⁷ cfu/ml. The control solution showed an average of 8.1×10⁶ cfu for the T-0 treatment interval, 4.8×10⁶ cfu for the T-7 treatment interval, and 8.5×10⁶ cfu for the T-24 treatment interval. The total remaining viable S. anginosus colonies after treatment with the 191 ppm nisin solution were 0.0 cfu for each of the T-0, T-7, and T-24 treatment intervals.

The S. anginosus control showed a less than 1 log decrease through 7 hours while returning to the original T-0 level by the T-24 timepoint. The Nisin-treated S. anginosus showed no detectable growth at the 0, 7, or 24 hour time points. It is possible that the test solution was not inoculated because nisin is not expected to show instantaneous kill as seen at the T-0 timepoint.

The results of this Example show that nisin is effective at reducing the levels of S. aureus, S. epidermidis, E. faecalis, P. acnes, and S. anginosus. The plate counts indicate that the nisin kill effectiveness was the greatest after 7 hours of treatment, with the exception of P. acnes, which had greater kill after 24 hours. Thus, it appears from the data that nisin begins to lose effectiveness, allowing for the regrowth of the organism over the full treatment interval. This suggests that greater effectiveness can be achieved by combining the nisin with other antimicrobials.

It is intended that the foregoing detailed description be regarded as illustrative, rather than limiting, and that it be understood that it is the following claims, including all equivalents, which are intended to define the scope of this invention.

We claim:

1. An antibiotic composition for decontaminating a biological tissue comprising:
a liquid solution comprising a lantibiotic in an amount effective to substantially inhibit bacterial growth of at least one type of gram-positive bacteria, wherein the solution is compatible with the biological tissue, such that when the solution is in contact with the biological tissue, the physiological characteristics of the biological tissue are substantially maintained.

2. The antibiotic composition of claim 1, wherein the lantibiotic comprises nisin.

3. The antibiotic composition of claim 1, wherein the solution further comprises at least one antifungal agent.

4. The antibiotic composition of claim 3, wherein the antifungal agent comprises amphotericin B.

5. The antibiotic composition of claim 1, wherein the lantibiotic is provided in an amount effective to substantially maintain the viability of the biological tissue.

6. The antibiotic composition of claim 1, further comprising at least one additional antimicrobial agent.

7. The antibiotic composition of claim 6, wherein the additional antimicrobial agent comprises vancomycin, imipenem, amikacin, or a combination thereof.

8. The antibiotic composition of claim 1, wherein the solution is at a pH between about 6 and about 8, and the lantibiotic is present at a concentration of from about 500 to 1200 IU/ml.

9. The antibiotic composition of claim 2, wherein the nisin is present at a concentration of about 1070 IU/ml.

10. The antibiotic composition of claim 1, wherein the at least one type of gram-positive bacteria comprises a bacteria selected from S. aureus, S. epidermidis, E. faecalis, P. acnes, C. sporogenes and S. anginosus.

11. A preservative for allograft and xenograft process solutions, comprising an effective amount of nisin in a physiological solution at a pH of between 3 and 8.

12. A method for decontaminating a biological tissue or cells comprising:
contacting the tissue or cells with an antibiotic composition comprising a lantibiotic for a period effective to substantially inhibit bacterial growth of at least one type of gram-positive bacteria.

13. The method of claim 12, wherein the lantibiotic comprises nisin.

14. The method of claim 12, wherein the lantibiotic is provided in an amount effective to maintain the physiological characteristics of the biological tissue or cells.

15. The method of claim 12, wherein the lantibiotic is provided in an amount effective to substantially maintain the viability of the biological tissue or cells.

16. A method for preparing an allograft tissue for transplantation comprising:
contacting the allograft tissue with an antibiotic composition comprising a lantibiotic for a period effective to substantially inhibit bacterial growth of at least one type of gram-positive bacteria.

17. The method of claim 16, wherein the lantibiotic comprises nisin.

18. The method of claim 16, wherein the allograft tissue is a heart valve, a blood vessel, pericardium or musculoskeletal tissue.

19. The method of claim 16, wherein the contacting step is performed at a temperature between about 2° C. and about 37° C.

20. The method of claim 16, wherein the period is between about 10 hours and about 48 hours.

21. The method of claim 16, wherein the antibiotic composition is a physiological solution further comprising a mixture of broad spectrum antimicrobial and antimycotic pharmaceutical agents.

22. The method of claim 21, wherein the physiological solution comprises vancomycin, imipenem, amikacin, and amphotericin B.

23. An aqueous solution comprising:
nisin in a concentration of about 1 mg per ml of solution or about 1070 IU per ml of solution;
vancomycin in a concentration of about 48 μg per ml of solution;
imipenem in a concentration of about 93 μg per ml of solution;
amikacin in a concentration of about 36 μg per ml of solution; and
amphotericin B in a concentration of about 4 μg per ml of solution.

24. The solution of claim 23, further comprising phosphate buffered saline.

25. A method for preparing an allograft tissue for transplantation comprising:
contacting the allograft tissue with a tissue compatible antibiotic composition for a period effective to substantially inhibit bacterial growth of at least one type of gram-positive bacteria by killing the at least one type of gram-positive bacteria under conditions wherein the at least one type of gram-positive bacteria is substantially metabolically inactive.

26. The method of claim 25, wherein the antibiotic composition comprises nisin.

27. A method for reducing bioburden on an allograft or xenograft tissue in coordination with a terminal sterilization procedure comprising:
contacting the allograft or xenograft tissue with an antibiotic composition comprising a lantibiotic for a period effective to substantially reduce the sterilization dose required in the terminal sterilization procedure to render the allograft or xenograft tissue sterile; and
sterilizing the allograft or xenograft tissue in the terminal sterilization procedure to render the allograft or xenograft tissue sterile.

28. The method of claim 27, wherein the terminal sterilization procedure comprises subjecting the allograft or xenograft tissue to ionizing radiation.

29. The method of claim 27, wherein the lantibiotic comprises nisin.
